(19) United States
(12) Patent Application Publication
(10) Pub. No.: US 2016/0151410 A1
(43) Pub. Date: Jun. 2, 2016

(54) CLEARANCE OF BIOACTIVE LIPIDS FROM MEMBRANE STRUCTURES BY CYCLODEXTRINS

(71) Applicants: Wanchao MA, New York, NY (US); Gaetano R. BARILE, River Vale,, NJ (US); David Choohyun PAIK, Cheltenham, PA (US)

(72) Inventors: Wanchao Ma, New York, NY (US); Gaetano R. Barile, River Vale, NJ (US); David Choohyun Paik, Cheltenham, PA (US)

(73) Assignee: The Trustees of Columbia University in the City of New York, New York, NY (US)

(21) Appl. No.: 14/900,556

(22) PCT Filed: Jun. 30, 2014
(86) PCT No.: PCT/US14/44919

§ 371 Date: Dec. 21, 2015

Related U.S. Application Data
(60) Provisional application No. 61/842,313, filed on Jul. 2, 2013.

(51) Int. Cl. A61K 31/724; A61K 9/00; A61K 45/06

(52) U.S. Cl. CPC A61K 31/724; A61K 45/06; A61K 9/0048

(57) ABSTRACT

The present invention provides a method of treating a subject suffering from wet acute macular degeneration which comprises administering to the subject an amount of a modified alpha cyclodextrin effective to treat the subject, wherein the modified alpha cyclodextrin binds to bioactive lipids which accumulate in the subject's eye and are characterized by the presence of a single chain of fatty acids.

Throughout this application, various publications are referenced. The disclosures of these publications are hereby incorporated by reference into this application to describe more fully the art to which this invention pertains.

### BACKGROUND OF THE INVENTION

Age-related macular degeneration (AMD) is a neurodegenerative eye disease associated with many risk factors, both environmental and genetic. AMD is the leading cause of vision loss in senior population of developed countries, and it is a major public health problem. (Friedman et al., 2004) There are two classic forms of AMD based upon whether there is growth of new blood vessels under the retinal pigment epithelium (RPE): neovascular and atrophic. The atrophic form is more common than the wet form, but it tends to progress more slowly than the wet form. It results from atrophy of photoreceptors and RPE cells without any abnormal vascularization. No medical or surgical treatment is available for this condition. The neovascular form of AMD is associated with the development of abnormal blood vessels (known as choroidal neovascularization) that usually proliferate under the RPE and often leads to more serious vision loss if untreated with anti-VEGF therapy or if associated with anatomic complications such as hemorrhage, fibrosis, and RPE rips.

Genetic studies have identified that the complement pathway and another locus, in chromosomal region 10q26, confer major susceptibility to the disease. (Edwards et al., 2005; Gold et al., 2006; Hageman et al., 2005; Haines et al., 2005; Klein et al., 2005; Rivera et al., 2005; Yates et al., 2007) Several additional loci with smaller odds ratios have also been shown to be associated with AMD, including apolipoprotein E (apoE) and hepatic lipase (LIPC). (Klaver et al., 1998; Neale et al., 2010; Souied et al., 1998) Smaller odds ratio and lower allele frequency attributable to certain genetic variant do not necessarily reflect the role of a gene and its encoded protein in the pathophysiology of disease. A single nucleotide polymorphism (SNP) can alter gene expression and/or its protein function, but the level of alteration can vary making it difficult to clarify the importance of a gene in a disease process by genetic studies alone. One of the best examples of this phenomenon can be seen in Alzheimer's disease with the discovery of the presenilin genes. (St George-Hyslop at al., 1992) While variation in the presenilin genes accounts for only several thousand Alzheimer's patients worldwide, the presenilin proteins are central to the processing of amyloid β, a hallmark of Alzheimer's pathogenesis and a target for drug therapy.

The presence of larger and more numerous drusen in the macula is the most common risk factor for AMD. A great effort to analyze drusen components aided the discovery of complement system activation in drusen. (Anderson et al., 2002; Hageman et al., 2001) Drusen also contains many other proteins and lipoprotein-like particles. (Ebrahimi and Handa, 2011; Mullins et al., 2000) In 2005, Y402H polymorphism of complement factor H (CFH) was reported to have strong association with AMD. (Edwards et al., 2005; Hageman at al., 2005; Haines et al., 2005; Klein et al., 2005) Thereafter, many studies further confirmed these initial reports. CFH is a regulator protein on complement activation; it competes with factor B for binding to C3b and functions as cofactor for the Factor I mediated C3b inactivation. The amino acid 402 of CFH is not involved in C3b binding, but it has been demonstrated that the AMD-associated 402H variant of CFH has lower binding affinity to C-reactive protein (CRP) than the 402Y variant. (Laine et al., 2007; Okemefuna et al., 2010) Patients with AMD and individuals who are homozygous for the CFH 402H allotype have increased level of CRP in drusen and basal deposits, (Bhutto et al., 2011; Johnson et al., 2006) but it is still unclear what molecules in drusen and basal deposit recruit CRP in these patients. The mechanisms for initiation of complement activation in drusen and the outer retina also remain unknown.

The present application investigates the nature of lysophospholipids in response to enzymatic modifications as they relate to known biological processes involved in AMD development. Specifically, the present application investigates the role of hepatic lipase in modifying lysophospholipids to become bioactive in complement activation, RPE cell death, and ocular neovascularization.

### SUMMARY OF THE INVENTION

The present invention provides a method of treating a subject suffering from wet acute macular degeneration which comprises administering to the subject an amount of a modified alpha cyclodextrin effective to treat the subject, wherein the modified alpha cyclodextrin binds to bioactive lipids which accumulate in the subject's eye and are characterized by the presence of a single chain of fatty acids.

The present invention also provides a method of treating a subject suffering from a cancer associated with lipid accumulation which comprises administering to the subject an amount of a modified alpha cyclodextrin effective to treat the subject, wherein the modified alpha cyclodextrin binds to the lipid.

The present invention also provides a method of treating a subject suffering from atherosclerosis associated with lipid accumulation which comprises administering to the subject an amount of a modified alpha cyclodextrin effective to treat the subject, wherein the modified alpha cyclodextrin binds to the lipid.

### BRIEF DESCRIPTION OF THE FIGURES

FIG. 1: LIPC-digested or alkaline-hydrolyzed human LDL or VLDL can activate the classical pathway of complement system.

96-well MaxiSorp plates were coated with fresh human LDL (A, D, E), VLDL (B), or NaOH-hydrolyzed LDL (C). After blocking with BSA, the plates, except (C), were digested with LIPC. To test complement activation, the plates were incubated with diluted native human serum (N), C1q-depleted human serum (Cd), factor B-depleted human serum (Bd), or human serum containing Mg-EGTA (ME). C3 fixation on the plate was detected with a monoclonal anti-C3d antibody followed by HRP-conjugated secondary antibody. Specific conditions for LIPC digestion and HRP-catalyzed color development of each experiment were outlined as following: LIPC was used at 10 μg/ml for (A, B, E), 0-50 μg/ml for (D), and no LIPC for (C). LIPC degradation was 1 hour for (D), 2 hours for (A, B), and 0-2 hours for (E). Color development was 15 minutes for (A, B, C, E) and 10 minutes for (D). For NaOH hydrolysis of LDL, 1.35 mg/ml of LDL was incubated in 0.15M NaOH solution at 25° C. for up to 60 minutes. Aliquots were taken out and neutralized with 0.15M HCl at 1, 10, 20, 60 minutes.

FIG. 2: Phospholipase A1 activity of LIPC and CEase is responsible for generation of complement-activating lipid molecules.

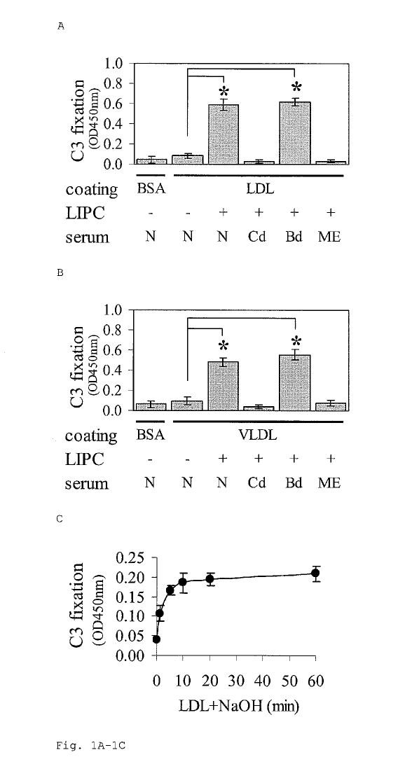

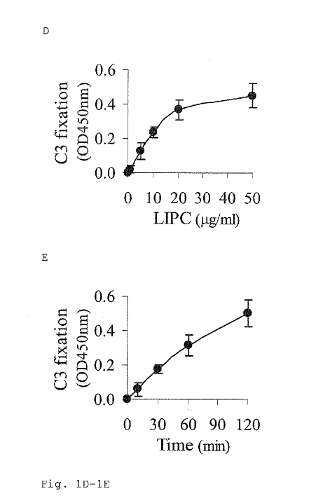

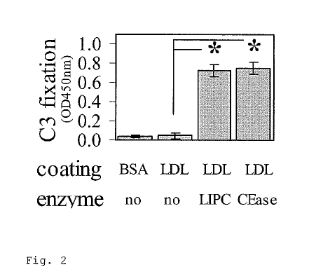

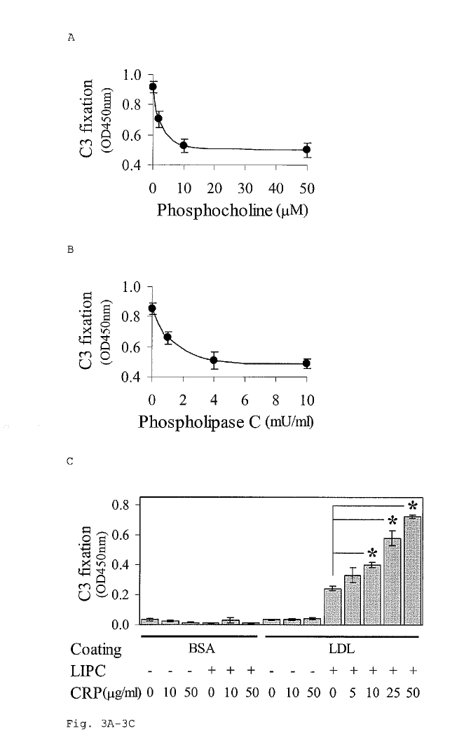

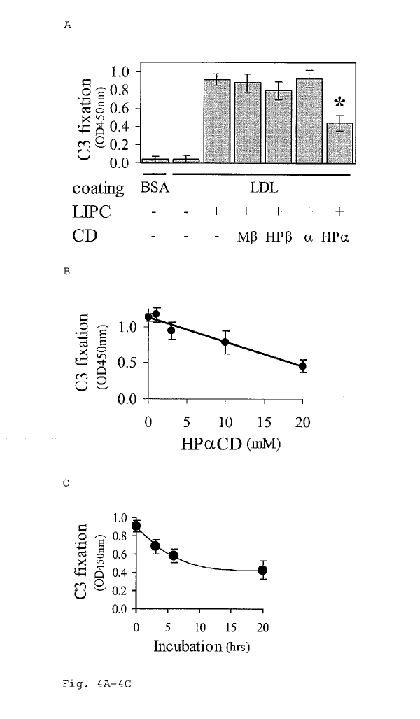

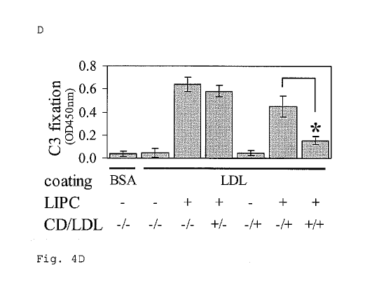

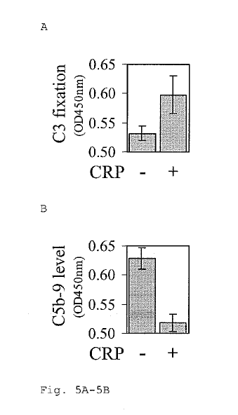

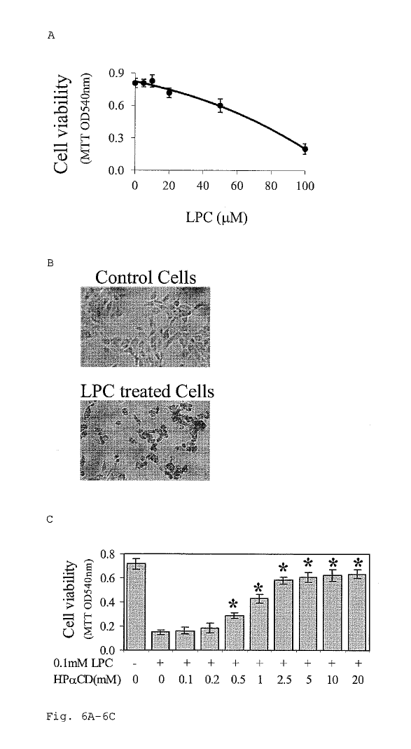

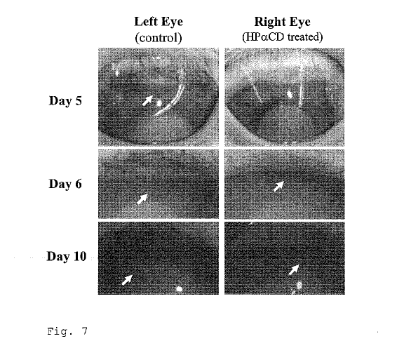

LIPC and CEase, both having 8 μUnits of phospholipase A1 activity but having 3 μUnits and 8125 μUnits of triglyceride hydrolase activity respectively, were used for 2 hours digestion of immobilized LDL. Complement activation with diluted native human serum is then determined as in FIG. 1. The HRP-catalyzed color development was 15 minutes.

FIG. 3: Lysophosphatidylcholine and CRP in the complement activation by LIPC-digested LDL.

Human LDL coating and 1 hour (C) or 2 hours (A, B) LIPC digestion was performed as described in FIG. 1. (A) For complement activation, 0-50 μM of phosphocholine was added into diluted native human serum. (B) LIPC-digested LDL was further treated with 0-10 mU/ml of phospholipase C in PBS containing 2% BSA and 5 mM CaCl₂ for 1 hour at 37° C., followed by complement activation. (C) Complement activation by LIPC-digested LDL was performed with diluted native human serum supplemented with purified human CRP. The HRP-catalyzed color development was 15 minutes for (A, B) and 10 minutes for (C).

FIG. 4: Extraction of bioactive lysophospholipids from LIPC-digested LDL by cyclodextrins (CD).

Immobilized LDL was digested with LIPC, then 20 mM of different cyclodextrins (A), or 0-20 mM HPαCD (B), or 20 mM HPαCD (C), or 2 mM of HPαCD in combination with 0.5 mg/ml of native LDL (D) are used for 20 hours extraction (A, B, D), or 0-20 hours extraction (C). Complement activation with diluted native human serum was then performed. MβCD—methyl-β-cyclodextrin; HPβCD—hydroxypropyl-β-cyclodextrin. Significant extraction of lysophospholipids by HPαCD was marked with * (A).

FIG. 5: Effect of additional CRP on early and terminal complement activation.

Immobilized LDL was digested by LIPC and then incubated with human serum with or without additional CRP. C3 fixation (A) and C5b-9 formation (B) was determined by specific primary and HRP-conjugated secondary antibodies.

FIG. 6: Cytotoxicity of 1-Palmitoyl-sn-glycero-3-phosphocholine on ARPE-19 cells.

(A, B) 40-50% confluent ARPE-19 cells in 96-well plate were cultured with serum-free medium for 24 hours, then incubated with 0.1 ml of serum-free medium containing 0-100 μM 1-Palmitoyl-sn-glycero-3-phosphocholine (LPC) for 22 hours. Cell morphology of control cells and 100 μM LPC treated cells was observed under a microscope, as shown in (B). The cells were then incubated with 0.15 ml of serum-free medium containing 0.5 mg/ml MTT at 37° C. for 2 hours. Formation of formazan is detected at 540 nm with 0.1 ml of DMSO as solvent (A). (C) As performed in (A), but with 100 μM LPC pre-incubated for 1 hour with 0-20 mM HPαCD, significant protection of HPαCD on ARPE-19 cells was observed when used at 0.5 mM or greater in medium (marked with *).

FIG. 7: HPαCD treatment on rabbit corneal neovascularization after alkali burn.

Alkali burn on central cornea was performed with 6 mm filter paper disc wetted with 8 μl of 1M NaOH. Cornea were then extensively washed with PBS, followed by treatment with control formula (left eye) and cyclodextrin formula (right eye) as described in Materials and Methods. The bottom two pictures are fluorescein angiograms showing the leaking new vessels in the cornea. White arrows in pictures show corneal neovascularization.

### DETAILED DESCRIPTION OF THE INVENTION

### Terms

As used in this application, except as otherwise expressly provided herein, each of the following terms shall have the meaning set forth below.

As used herein, the term “administering” may be effected or performed using any of the methods known to one skilled in the art. The methods comprise, for example, intralesional, intramuscular, subcutaneous, intravenous, intraperitoneal, liposome-mediated, transmucosal, intestinal, topical, nasal, oral, anal, ocular or otic means of delivery.

As used herein, the term “composition”, as in pharmaceutical composition, is intended to encompass a product comprising the active ingredient(s) and the inert ingredient(s) that make up the carrier, as well as any product which results, directly or indirectly from combination, complexation, or aggregation of any two or more of the ingredients, or from dissociation of one or more of the ingredients, or from other types of reactions or interactions of one or more of the ingredients.

As used herein, “effective amount” refers to an amount which is capable of treating a subject having a tumor, a disease or a disorder. Accordingly, the effective amount will vary with the subject being treated, as well as the condition to be treated. A person of ordinary skill in the art can perform routine titration experiments to determine such sufficient amount. The effective amount of a compound will vary depending on the subject and upon the particular route of administration used. Based upon the compound, the amount can be delivered continuously, such as by continuous pump, or at periodic intervals (for example, on one or more separate occasions). Desired time intervals of multiple amounts of a particular compound can be determined without undue experimentation by one skilled in the art. In one embodiment, the effective amount is between about 1 μg/kg-10 mg/kg. In another embodiment, the effective amount is between about 10 μg/kg-1 mg/kg. In a further embodiment, the effective amount is 100 μg/kg.

“Inhibiting” the onset of a disorder or undesirable biological process shall mean either lessening the likelihood of the disorder's or process' onset, or preventing the onset of the disorder or process entirely. In the preferred embodiment, inhibiting the onset of a disorder or process means preventing its onset entirely.

As used herein, a “modified alpha cyclodextrin” is an alpha cyclodextrin in which one or more of the hydrogen atoms of the hydroxyl moieties present on carbons 2, 3 and 6 of the alpha cyclodextrin subunits are substituted with a moiety other than hydrogen.

Table 1 presents examples of modified α-cyclodextrins and examples of substituents thereon.

TABLE 1
Examples of modified α-cyclodextrins

| | |
|---|---|
| Methyl | —CH₃ |
| Ethyl | —CH₂CH₃ |
| Butyl | —CH₂CH₂CH₂CH₃ |
| Acetyl | —COCH₃ |
| 6-t-butyldimethylsilyl | |
| 2,3-dimethyl-6-t-butyldimethylsilyl | |
| 2,6-diacetyl-3-t-butyldimethylsilyl | |
| 2,6-dibutyldimethylsilyl | |
| 2,6-dimethyl-3-pentyl | |
| 2,6-dipentyl | —CH₂CH₂CH₂CH₂CH₃ |
| 2,6-dipentyl-3-acetyl | |
| 2,6-dipentyl-3-butyl | |
| 2,6-dipentyl-3-trifluoroacetic | |
| Maltosyl | |
| Peracetyl Maltosyl | |
| Carboxyethyl | —CH₂CH₂COOH |
| (2-Cyano)ethyl | —CH₂CH₂CN |
| Hydroxybutyl | —CH₂CH(OH)CH₂CH₃ |
| Hydroxyethyl | —CH₂CH₂OH |
| Hydroxypropyl | —CH₂CH(OH)CH₃ |
| Sulfate | —SO₃H |
| Phosphate | —PO(OH)2 |
| Sulfobutyl | —CH₂CH₂CH₂CH₂SO₃H |
| Sulfopropyl | —CH₂CH₂CH₂SO₃H |
| Succinyl | —COCH₂CH₂COOH |
| Succinylhydroxypropyl | —COCH₂CH₂COOCH₂CH(OH)CH₃ |
| 2,3,6-Triacetyl (insol.) | —COCH₃ |
| Triacetyl (insol.) | —COCH₃ |
| Tribenzoyl | —COC₆H₅ |
| 2,3,6-Triethyl | —CH₂CH₃ |
| 2,3,6-TriMethyl | —CH₃ |
| Trioctyl | —CH₂CH₂CH₂CH₂CH₂CH₂CH₂CH₃ |
| Tri (trifluoroacetic) | —COCF₃ |

As used herein, “pharmaceutically acceptable carrier” means that the carrier is compatible with the other ingredients of the formulation and is not deleterious to the recipient thereof, and encompasses any of the standard pharmaceutically accepted carriers. Such carriers include, for example, 0.01-0.1 M and preferably 0.05 M phosphate buffer or 0.8% saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like.

“Subject” shall mean any organism including, without limitation, a mammal such as a mouse, a rat, a dog, a guinea pig, a ferret, a rabbit and a primate. In one embodiment, the subject is a human.

“Treating” means either slowing, stopping or reversing the progression of a disease or disorder. As used herein, “treating” also means the amelioration of symptoms associated with the disease or disorder.

Units, prefixes and symbols may be denoted in their SI accepted form.

### EMBODIMENTS OF THE INVENTION

The present invention provides a method of treating a subject suffering from wet acute macular degeneration which comprises administering to the subject an amount of a modified alpha cyclodextrin effective to treat the subject, wherein the modified alpha cyclodextrin binds to bioactive lipids which accumulate in the subject's eye and are characterized by the presence of a single chain of fatty acids.

In one or more embodiments, the binding of the modified alpha cyclodextrin to the bioactive lipids facilitates clearance of the lipids from the subject's eye.

The method of claim 1 or 2, wherein the modified alpha cyclodextrin is selected from the group consisting of hydroxypropyl alpha cyclodextrin, hydroxybutyl alpha cyclodextrin, sulfobutyl alpha cyclodextrin, sulfopropyl alpha cyclodextrin, carboxyethyl alpha cyclodextrin, succinyl alpha cyclodextrin and succinylhydroxypropyl alpha cyclodextrin.

In one or more embodiments, the modified alpha cyclodextrin is selected from the group consisting of 2-hydroxypropyl alpha cyclodextrin, 2-hydroxybutyl alpha cyclodextrin and 2-succinylhydroxypropyl alpha cyclodextrin.

In one or more embodiments, the modified alpha cyclodextrin is 2-hydroxypropyl alpha cyclodextrin.

In one or more embodiments, the bioactive lipids are lysophospolipids.

In one or more embodiments, the modified alpha cyclodextrin is administered as a monotherapy.

In one or more embodiments, the method further comprises coadministering a second therapeutic agent for treating acute macular degeneration.

In one or more embodiments, the second therapeutic agent is selected from the group consisting of ranibizumab, bevacizumab, pegaptanib sodium, aflibercept and verteporfin.

In one or more embodiments, the administering comprises administering eyedrops to the subject.

In one or more embodiments, the administering comprises intravitreally injecting the modified alpha cyclodextrin.

The present invention also provides a method of treating a subject suffering from a cancer associated with lipid accumulation which comprises administering to the subject an amount of a modified alpha cyclodextrin effective to treat the subject, wherein the modified alpha cyclodextrin binds to the lipid.

In one or more embodiments, the lipid is characterized by the presence of a single chain of fatty acids.

In one or more embodiments, the modified alpha cyclodextrin is selected from the group consisting of hydroxypropyl alpha cyclodextrin, hydroxybutyl alpha cyclodextrin, sulfobutyl alpha cyclodextrin, sulfopropyl alpha cyclodextrin, carboxyethyl alpha cyclodextrin, succinyl alpha cyclodextrin and succinylhydroxypropyl alpha cyclodextrin.

In one or more embodiments, the modified alpha cyclodextrin is selected from the group consisting of 2-hydroxypropyl alpha cyclodextrin, 2-hydroxybutyl alpha cyclodextrin and 2-succinylhydroxypropyl alpha cyclodextrin.

In one or more embodiments, the modified alpha cyclodextrin is 2-hydroxypropyl alpha cyclodextrin.

In one or more embodiments, the lipids comprise lysophospolipids.

In one or more embodiments, the modified alpha cyclodextrin is administered as a monotherapy.

In one or more embodiments, the method further comprises coadministering a second therapeutic agent for treating cancer.

In one or more embodiments, the second therapeutic agent is selected from the group consisting of temozolomide, a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, camptothecins, bleomycin, doxorubicin, idarubicin, daunorubicin, dactinomycin, plicamycin, mitoxantrone, L-asparaginase, epirubicin, 5-fluorouracil, taxanes such as docetaxel and paclitaxel, leucovorin, levamisole, irinotecan, estramustine, etoposide, nitrogen mustards, 1,3-bis(2-chloroethyl)-1-nitrosourea (BCNU), nitrosoureas such as carmustine and lomustine, vinca alkaloids such as vinblastine, vincristine and vinorelbine, platinum complexes such as cisplatin, carboplatin and oxaliplatin, imatinib mesylate, hexamethylmelamine, topotecan, tyrosine kinase inhibitors, tyrphostins, herbimycin A, genistein, erbstatin and lavendustin A.

The present invention also provides a method of treating a subject suffering from atherosclerosis associated with lipid accumulation which comprises administering to the subject an amount of a modified alpha cyclodextrin effective to treat the subject, wherein the modified alpha cyclodextrin binds to the lipid.

In one or more embodiments, the lipid is characterized by the presence of a single chain of fatty acids.

In one or more embodiments, the modified alpha cyclodextrin is selected from the group consisting of hydroxypropyl alpha cyclodextrin, hydroxybutyl alpha cyclodextrin, sulfobutyl alpha cyclodextrin, sulfopropyl alpha cyclodextrin, carboxyethyl alpha cyclodextrin, succinyl alpha cyclodextrin and succinylhydroxypropyl alpha cyclodextrin.

In one or more embodiments, the modified alpha cyclodextrin is selected from the group consisting of 2-hydroxypropyl alpha cyclodextrin, 2-hydroxybutyl alpha cyclodextrin and 2-succinylhydroxypropyl alpha cyclodextrin.

In one or more embodiments, the modified alpha cyclodextrin is 2-hydroxypropyl alpha cyclodextrin.

In one or more embodiments, the lipids comprise lysophospolipids.

In one or more embodiments, the modified alpha cyclodextrin is administered as a monotherapy.

In one or more embodiments, the method further comprises coadministering a second therapeutic agent for treating atherosclerosis.

In one or more embodiments, the second therapeutic agent is selected from the group consisting of HMG-CoA reductase inhibitors (statins), fibric acid derivatives, bile acid sequestrants, cholesterol absorption inhibitors and niacin.

As used herein, “about” with regard to a stated number encompasses a range of +10 percent to −10 percent of the stated value. By way of example, about 100 mg/kg therefore includes the range 90-110 mg/kg and therefore also includes 90, 91, 92, 93, 94, 95, 96, 97, 98, 99, 100, 101, 102, 103, 104, 105, 106, 107, 108, 109 and 110 mg/kg. Accordingly, about 100 mg/kg includes, in an embodiment, 100 mg/kg.

It is understood that where a parameter range is provided, all integers within that range, tenths thereof, and hundredths thereof, are also provided by the invention. For example, “0.2-5 mg/kg” is a disclosure of 0.2 mg/kg, 0.21 mg/kg, 0.22 mg/kg, 0.23 mg/kg etc. up to 0.3 mg/kg, 0.31 mg/kg, 0.32 mg/kg, 0.33 mg/kg etc. up to 0.4 mg/kg, 0.5 mg/kg, 0.6 mg/kg etc. up to 5.0 mg/kg.

All combinations of the various elements described herein are within the scope of the invention.

This invention is illustrated in the Experimental Details section which follows. This section is set forth to aid in an understanding of the invention but is not intended to, and should not be construed to, limit in any way the invention as set forth in the claims which follow thereafter.

### EXPERIMENTAL DETAILS

Methods

Materials

Two recombinant human LIPC were purchased from GeneTex (GTX48178-PRO, Irvine, Calif.) and OriGene (TP315870, Rockville, Md.). Cholesterol esterase (CEase) was obtained from MP Biomedicals (0210543950, Solon, Ohio). 1,2-bis(heptanoylthio)glycerophosphocholine and phosphocholine from Santa Cruz Biotechnology (Santa Cruz, Calif.). Human LDL, phospholipase C, human CRP, 1-Palmitoyl-sn-glycero-3-phosphocholine, 2,3-dimercapto-1-propanol tributyrate (DMPTB), and bovine serum albumin (BSA) prepared by heat shock fractionation were obtained from Sigma Aldrich (St. Louis, Mo.). Human VLDL was Kalen Biomedical (Montgomery Village, Md.) a product. Normal human serum, C1q-depleted human serum, factor B-depleted human serum, and a monoclonal antibody to a neoepitope in the C3d domain of C3 were obtained from Quidel (San Diego, Calif.). Monoclonal mouse anti-human C5b-9 was purchased from Dako (Carpinteria, Calif.). All cell culture products were from Life Technologies (Grand Island, N.Y.). POPC liposome was obtained from AbboMax (San Jose, Calif.).

Animals

All of the experiments involving rabbits were approved by the Institutional Animal Care and Use Committee of Columbia University and complied with guidelines set forth by the Association for Research in Vision and Ophthalmology (ARVO) Statement for the Use of Animals in Ophthalmic and Vision Research.

Human LDL and VLDL Degradation and Complement Activation

96-well NUNC MaxiSorp plates were coated with 40 μl of human LDL, VLDL, or NaOH-hydrolyzed LDL, all at 200 μg/ml in PBS, at 4° C. overnight then 37° C. for 1 hour, and remaining binding sites were blocked with 3% BSA in PBS at 37° C. for 1 hour. The wells were washed and then incubated with 40 μl of LIPC or CEase in PBS containing 2% BSA at 37° C. for 1 or 2 hours as indicated in Results section, and the degradation reaction was stopped by washing the plate with PBS. For complement system activation, the resulted plate was incubated with 20 μl of 1:1 diluted human sera at 21° C. for 30 minutes. The diluent for serum dilution was PBS containing calcium and magnesium, and human sera that were utilized in our study were native serum, C1q-depleted serum, factor B-depleted serum, native serum with addition of 10 mM of MgCl₂ and 20 mM EGTA (Mg-EGTA), and native serum with addition of 10 mM EDTA.

After thorough washing of the plate, C3 fixation and final membrane attack complex formation on the plate were determined with anti-human C3d antibody and anti-human C5b-9 antibody, in combination with HRP-conjugated secondary antibody, respectively. The monoclonal anti-human C3d antibody is reactive to all C3d-containing fragments of C3, but not with C3 itself, so it detects C3 fixation on the plate and not C3 absorption. The final peroxidase activity was monitored at 450 nm with 3,3′,5,5′-tetramethyl-benzidine and hydrogen peroxide as substrates after 10 or 15 minutes reaction at room temperature. Addition of EDTA into native human serum totally blocked complement activation; thus it was used as control for complement activation studies and was set as baseline for every experiment.

Cell Culture and Cell Viability Assay

Human retinal pigment epithelial cell line, ARPE-19, was purchased from ATCC (Manassas, Va.). The cells were cultured in Dulbecco's modified Eagle's medium supplemented with 10% of heat-inactivated fetal bovine serum (FBS), 100 IU/ml of penicillin, and 100 ng/ml of streptomycin (all cell culture products from Invitrogen-Gibco, Rockville, Md.). Cells were maintained at 37° C. in a 5% CO₂ incubator with medium change every 3-4 days. Subculture of ARPE-19 cells was performed with 0.05% trypsin-EDTA solution.

Cell viability was determined with the colorimetric MTT metabolic activity assay. ARPE-19 cells cultured in 96-well cluster plates were incubated with 0.15 ml of serum-free medium containing 0.5 mg/ml of MTT at 37° C. for 2 hours in a 5% CO₂ incubator. After removal of cell culture medium, formation of formazan from MTT reduction was detected at 540 nm with 0.1 ml of DMSO as solvent.

Enzymatic Activities

Triglyceride hydrolase activity of CEase and LIPC was determined with DMPTB as substrate according to Choi et al. (Choi et al., 2003) Phospholipase A activity of CEase and LIPC was assayed similarly as triglyceride hydrolase activity but with 1.5 mM of 1,2-bis(heptanoylthio)glycerophosphocholine as substrate and assay buffer containing 10 mM of CaCl₂ instead of 1 mM of EDTA.

Rabbit Corneal Neovascularization by Alkali Burn

3 New Zealand white rabbits, aged 4-5 months and weighing 1.8-2.6 kg, were obtained from Charles River Laboratories (Wilmington, Mass.) and acclimatized for several weeks before the experiments started. All of the rabbits were anesthetized using ketamine (42 mg/kg) and xylazine (7 mg/kg) intramuscularly. 0.5% proparacaine hydrochloride (Akorn, Inc. Lake Forest, Ill.) was applied topically. Alkali burn on central cornea of both eyes was performed with 6 mm round disc of Whatman No. 1 filter paper wetted with 84 of 1M NaOH for exactly 1 minute, filter paper was then removed and the cornea was extensively irrigated with 200 ml of PBS for 10 minutes. Left eye and right eye were then treated with control formula (6 mg/ml of POPC liposome in PBS) and cyclodextrin formula (50 mM HPαCD and 16.7 mM HPβCD in control formula), respectively. Both eyes were first treated with 0.5 ml of respective formula that were hold in a 90 mm Hessburg-Barron Vacuum Trephine placed on top of the alkali-burned cornea for 1 hour, and then followed by eye drops every half hour for 4 hours. The same treatment was repeated on day 2, and then only eye drops were applied every hour from day 3 to day 5 for 8 hours each day. No further treatment was applied after day 5.

Statistical Analysis

All values are presented as mean±SD. Where applicable, a 2-tailed Student's t-test was employed to analyze the statistics of two groups (Excel software; Microsoft, Redmond, Wash.). Minimum values of P less than 0.01 were considered as statistically significant and denoted by * in each Figures.

### EXAMPLES

### Example 1

### LIPC Degradation of LDL and VLDL Generates Lipid Molecules that can Activate Classical Complement Pathway

LIPC degradation of either human lipoproteins LDL or VLDL caused these lipoproteins to biologically activate the complement system (FIG. 1). LIPC degradation of LDL and VLDL is through a calcium independent mechanism (data not shown). The complement activation is both dose and time-dependent upon LIPC degradation (FIGS. 1D and 1E, respectively). When either C1q-depleted serum or Mg-EGTA-containing serum was used, complement activation did not occur, indicating that the classical pathway is involved. When factor B depleted serum was used, there was no change in the level of complement activation, indicating that the alternative pathway is not involved. Two LIPC products were compared (see Materials) upon degradation of human lipoproteins, and both enzymes had similar activities for initiating lipoproteins to activate the complement system (data not shown). LIPC from GeneTex was employed for most of the experiments described in the present application.

Saponification of lipids is a well-known process that produces soap. Like LIPC digestion, mild alkaline hydrolysis of phospholipids can generate lysophospholipids and fatty acids. (Kensil and Dennis, 1981) We tested the alkaline-hydrolyzed LDL with human serum—as shown in FIG. 10, alkaline hydrolysis of LDL can quickly generate lipid molecules that activate complement system.

### Example 2

### Lysophosphatidylcholine and its Binding Protein CRP Play Major Roles in Complement Activation

Additional studies using an enzymatic activity analysis show that lysophospholipids have major roles in complement activation. Cholesterol esterase (CEase) has a broad spectrum of substrates that include triglycerides, phospholipids, cholesterol esters, and lipoproteins. Similar to what was observed with LIPC-digested LDL, CEase-digested LDL is known to activate the complement system via the classical pathway. (Biro et al., 2007) Although both LIPC and CEase have both phospholipase A1 and triglyceride hydrolase activity, their proportional activities vary. Specifically, when equivalent phospholipase A1 activity is present for both CEase and LIPC, CEase has much greater triglyceride hydrolase activity than LIPC. As shown in FIG. 2, similar levels of complement activation were observed by utilizing equivalent phospholipase A1 activity, 8 μunits, of both LIPC and CEase, when the triglyceride hydrolase activity has >2700-times difference (Table 2). This suggests that phospholipase A1 activity is the primary enzymatic activity that generates a complement-activating lipid species in the setting of these enzymes. Triglyceride hydrolase is known to digest LDL into fatty acids, monoglycerides and diglycerides, while phospholipase A1 is known to digest LDL into fatty acids and lysophospholipids. Thus we can infer that not only is phospholipase activity responsible for complement activation but that lysophospholipids mediate this effect (i.e. are the complement activating lipid species in these settings).

TABLE 2
PLA1 and TG hydrolase activity of LIPC and Cease

| Enzyme Activity | LIPC | CEase |
|---|---|---|
| PLA1 (μUnits) | 8 | 8 |
| TGhydrolase (μUnits) | 3 | 8125 |

Phosphatidylcholine is the most abundant phospholipid in cell membranes and lipoproteins, helping to maintain the structure of the membrane bilayer. It might be expected that the major lysophospholipid on LIPC-degraded lipoproteins is lysophosphatidylcholine, a well-studied ligand for CRP in membrane structures. (Volanakis and Wirtz, 1979) Immobilization of CRP can activate classical complement pathway by interaction with C1, so additional experiments were focused on lysophosphatidylcholine. When phosphocholine is added into native human serum as a competitive binding inhibitor for CRP, (Volanakis and Narkates, 1981) it significantly decreases complement activation (FIG. 3A). Phospholipase C, which specifically hydrolyzes the phosphorylcholine group in lysophosphatidylcholine, also demonstrates a significant treatment effect in reducing complement activation (FIG. 3B). In further experiments, BSA alone or treated with LIPS did not activate the complement system, nor did native LDL. Raising the CRP level to that of native human serum did not alter the activity of these molecules to initiate complement activation. But LIPC-digested LDL can induce C3 fixation, and the addition of CRP dose-dependently enhances its activity on complement activation (FIG. 3C).

### Example 3

### Extraction of Lysophospholipids from LIPC-Digested Lipoprotein by Cyclodextrins

We tested several cyclodextrins for the ability to extract lysophospholipids from LIPC-digested human LDL. Since lysophospholipid level in lipoprotein is directly related to complement C3 fixation, we use C3 fixation as an indicative parameter for levels of lysophospholipids in lipoprotein. As shown in FIG. 4A, incubation of LIPC-digested LDL with 2-hydroxypropyl-α-cyclodextrin (HPαCD) resulted in significantly less complement activation, while other cyclodextrins, including β-cyclodextrin (data not shown), methyl-β-cyclodextrin, 2-hydroxypropyl-β-cyclodextrin, and α-cyclodextrin, had much weaker effects. HPαCD extraction of complement-activating lysophospholipids is dose- and time-dependent (FIG. 4B, 4C). We also tested HPαCD upon shuttling of lysophospholipids between LIPC-digested LDL and native LDL, a biological feature of lipid transport (FIG. 4D). Low levels of HPαCD alone or native LDL alone cannot extract much lysophospholipids, but in combination, they extract the most lysophospholipids from LIPC-digested LDL. HPαCD thus displays a shuttle function for lysophospholipids.

### Example 4

### Involvement of CFH in the Complement Activation

A unique feature of CRP-induced complement activation is that the complement activation is restricted to early complement components, while the formation of more damaging terminal complement complex is minimal. Such activity of CRP is the result of CRP recruitment of factor H. (Mold et al., 1999) Amino acid change of 402Y to 402H reduces factor H binding affinity for CRP, so it could be expected that, when serum with the 402H variant of factor H is used in complement activation studies, LIPC-digested lipoproteins will generate more terminal complex. Addition of pure human CRP molecules into native human serum limits terminal complement complex formation. As shown in FIG. 5, addition of CRP enhances C3 fixation induced by LIPC-digested LDL, but the same CRP blocks the terminal complement complex formation, indicating CFH is involved in such system. A more dramatic difference on the formation of terminal complement complex could be expected when human serum containing 402H CFH is available.

### Example 5

### Lysophosphatidylcholine is Cytotoxic to RPE Cells

The effects of lysophosphatidylcholine with regard to cytotoxic activity against RPE cells was assessed. 1-palmitoyl-sn-glycero-3-phosphocholine is one of the most predominant lysophospholipid products resulting from the hydrolysis of biological membrane and is a molecule with known cytotoxic activity to many different types of cells. As shown in FIGS. 6A and B, when 1-palmitoyl-sn-glycero-3-phosphocholine was added to the cell culture medium at 20 μM or greater, it induced ARPE-19 cell death. Pre-incubation of lysophosphatidylcholine with HPαCD effectively attenuated this cytotoxic activity (FIG. 6C).

### Example 6

### Lysophospholipids Induce Ocular Neovascularization

The alkali burn model was utilized in our study of lysophospholipids in rabbit corneal neovascularization. Alkali burns of the cornea generate a large amount of lipid mixtures containing fatty acids and lysophospholipids. These lipid mixtures can form small micelles and vesicles that diffuse along the collagen fibers in the corneal stroma, and they can be further processed by other corneal cells to generate new bioactive lipids. As one example, lysophosphatidylcholine can be used to generate lysophosphatidic acid (LPA) and platelet-activating factor (PAF). HPαCD extraction applied after alkali burn can reduce the amount of the bioactive lipids in the stroma. If these bioactive lipids have roles in neovascularization, their removal will show an impact on corneal neovascularization. Three rabbits have been tested thus far. All of them showed dramatic effect of HPαCD extraction on inhibition of corneal neovascularization. FIG. 7 shows a representative result that both the neovascularization area and vessel length are reduced dramatically. None of the rabbits shows any signs of cyclodextrin toxicity. Conjunctival vessels also showed dramatic differences between control eyes and cyclodextrin-treated eyes with treated eyes exhibiting much less hyperemia. The conjunctival vessels support the ingrowth of corneal neovessels, and a direct relationship could be observed in all the eyes between the amount of neovessels observed in the cornea and hyperemia in conjunctiva.

Consistent with published literature, corneal thickness also was noted to increase dramatically following the alkali burn injury, returning to normal thickness levels within about one week. A second phase of swelling then occurred and lasted for weeks. The first corneal edema phase is felt to be the result of corneal epithelial and endothelial cells loss. Regrowth and functional recovery of these cells over the course of 1 week results in the normalization of corneal thickness (via a water pumping mechanism). Cyclodextrins/liposome treatment showed enhanced functional recovery of corneal epithelial and endothelial cells in the first corneal edema phase in all three rabbits. In all three animals the corneal thickness normalized faster than controls (see Table 3). Because it is well-known that lysophosphatidylcholine can directly increase endothelial permeability by inducing endothelial cell contraction and by decreasing tight junction proteins expression (Wang at al.; 2009; Barile at al. 1999; Barile at al. 2005), the effect of cyclodextrins/liposome treatment on resolving the first phase of corneal edema provides additional evidence that cyclodextrins are capable of removing lysophosphatidylcholine.

According to chemical structure similarities, the following alpha cyclodextrins should have similarly effective drug activity as hydroxypropyl-a-cyclodextrin: hydroxybutyl, carboxyethyl, sulfobutyl, sulfopropyl, succinyl, succinylhydroxypropyl.

TABLE 3
Rabbit central corneal thickness measured by ultrasonic
pachymeter

| | | Central corneal thickness (micron) | | | | | |
|---|---|---|---|---|---|---|---|
| Rabbit | Eye | Day 0 | Day 3 | Day 4 | Day 5 | Day 6 | Day 7 |
| 1 | left | 343 | 1025 | 817 | 545 | | |
| | right | 351 | 1020 | 716 | 386 | | |
| 2 | left | 371 | 1030 | 1031 | 917 | 887 | 842 |
| | right | 367 | 1030 | 1034 | 902 | 768 | 667 |
| 3 | left | 354 | 1028 | 1020 | 893 | 693 | 654 |
| | right | 351 | 1022 | 1026 | 892 | 572 | 344 |

### DISCUSSION

In the present application, we have identified a molecular linkage among drusen components and several well-defined biological processes in the development of AMD, including complement activation, RPE cell death, and neovascularization. Our experimental data indicate that the enzyme product of at least one AMD susceptibility gene, LIPC, can initiate the generation of bioactive lysophospholipids by hydrolyzing lipid components of the outer retina. CRP binding to lysophosphatidylcholine activates the classical pathway of complement systems, while CFH binding to CRP plays a central role in inhibiting such complement activation at the C3b level. The 402H variant of CFH loses its regulatory activity on complement activation due to its weak binding to CRP, leading to the completion of complement activation.

Fixation of early complement components is normally utilized for the safe clearance of cell debris and apoptotic cells through phagocytosis, while the membrane attack complex formation will generate inflammatory activity at the outer retinal layers. We also demonstrated that lysophospholipid itself was cytotoxic to RPE cells and could lead to RPE cell death. In consideration of both complement activation and the cytotoxicity of lysophospholipids, RPE cells that are in close proximity to drusen and basal deposits could be one target cell of lysophospholipids.

Lysophospholipids generated by LIPC hydrolysis of lipoproteins can be further processed by retinal cells, specifically photoreceptors, RPE cells and choroidal vascular cells, to generate additional bioactive lysophospholipids, such as LPA and sphingosine-1-phosphate (S1P). LPA and S1P may only account for a small portion of the whole lysophospholipid pool, but their biological activities are dominant in angiogenesis. (Houben and Moolenaar, 2011; Moolenaar and Hla, 2012) We utilized a rabbit alkali burn corneal neovascularization model to study the roles of lysophospholipids in angiogenesis, and when HPαCD was used to clear the lysophospholipids generated by alkaline hydrolysis of corneal phospholipids, it showed dramatic inhibition on corneal neovascularization.

A common feature early in the pathogenesis of AMD is deposit formation in the region of the RPE and Bruch's membrane interface. Depositing material in Bruch's membrane results in progressive Bruch's membrane thickening and the appearance of drusen, which is a clinical marker for the disease. About 40% of these deposits consist of lipids in the form of lipoprotein-like particles that contain apoA-I, apoB-100, apoE, apoC-I and apoC-II. (Li et al., 2006; Wang at al., 2010) Lipid profiles of such deposits have shown high levels of lysophospholipids and free fatty acids, suggesting that the hydrolysis of phospholipids, such as phosphatidylcholine, has occurred. (Curcio et al., 2010; Wang et al., 2009) Furthermore, transmission electron micrographs of the lipoprotein-like particles accumulating underneath RPE cells in AMD patient show a morphology that is consistent with a model of surface degradation and particle fusion. (Curcio et al., 2011) Retention, or trapping, of lipoprotein-like particles will certainly subject these particles to oxidation and degradation by local enzymes released from RPE cells, such as LIPC, lipoprotein lipase, and secretory phospholipase A2, or enzyme that is carried over by lipoproteins from blood, such as lipoprotein-associated phospholipase A2.

A human RPE cell culture model that mimics early stage of AMD with accumulation of sub-RPE deposits has shown that the deposits consist of two morphologically distinct forms of deposits: One consisting of membrane-bounded multivescicular material, and the other of nonmembrane-bounded particle conglomerates. (Johnson et al., 2011) When exposed to human serum, the deposits can trigger complement activation that appears to be mediated via the classical pathway by binding of C1q to ligands in apoE-rich deposits specifically. IgG depletion has no detectable effect on complement activation in comparison with whole serum controls, thus suggesting that activation of the classical pathway occurs via an antibody-independent mechanism. The exact C1q binding partners were not identified in that study. Based upon our studies, it might be expected that phospholipases released from human RPE cells will degrade apoE-containing membrane deposits and generate lysophospholipids that can initiate antibody-independent classical pathway activation.

A collaborative genome-wide association study, including >17,100 advanced AMD cases and >60,000 controls of European and Asian ancestry, identified 19 loci that associated with AMD at P<5×10⁻⁸. (Fritsche et al., 2013) To identify biological relationships among these genetic association signals, the genes within 100 kb of the variants in each association peak were analyzed, and total of 90 genes were obtained when correlation was set at r²>0.8. Ingenuity Pathway Analysis highlighted several biological pathways, particularly the complement system, atherosclerotic signaling, and angiogenesis, that were enriched in the resulting set of 90 genes. Interestingly, phospholipid degradation was identified as top 5 pathway with nominal P value at 0.0058. Three genes were in this pathway: PLA2G12A, LIPC, PLA2G6. PLA2G12A is a secretory phospholipase A2, while PLA2G6 is a cytosolic calcium-independent phospholipase A2. Compared with single-gene or single-SNP analyses, gene set/pathway association analyses can potentially reduce the false positives and uncover a significant biological effect distributed over multiple loci even if changes in any individual locus have a small effect. As an example, each of the three genes in phospholipid degradation pathway has weak association with AMD, but together they form a pathway that has strong association with AMD.

Lysophospholipids are major component of oxLDL, (many references, lipoprotein-associated phospholipase A2 is involved) subretinal injection of oxLDL induced choroidal neovascularization in mice. (Proc Natl Acad Sci USA. 2012 Aug. 21; 109(34):13757-62) Oxidative stress is one of AMD risk factors, trapped lipoprotein-like particles under RPE are subjected to such stress. Oxidized phospholipids are substrates for lipoprotein-associated phospholipase A2.

### REFERENCES

Anderson, D. H., Mullins, R. F., Hageman, G. S., Johnson, L. V., 2002. A role for local inflammation in the formation of drusen in the aging eye. Am J Ophthalmol 134, 411-431.
Bhutto, I. A., Baba, T., Merges, C., Juriasinghani, V., McLeod, D. S., Lutty, G. A., 2011. C-reactive protein and complement factor H in aged human eyes and eyes with age-related macular degeneration. Br J Ophthalmol 95, 1323-1330.
Biro, A., Thielens, N. M., Cervenak, L., Prohaszka, Z., Fust, G., Arlaud, G. J., 2007. Modified low density lipoproteins differentially bind and activate the C1 complex of complement. Mol Immunol 44, 1169-1177.
Choi, S. J., Hwang, J. M., Kim, S. I., 2003. A colorimetric microplate assay method for high throughput analysis of lipase activity. J Biochem Mol Biol 36, 417-420.
Curcio, C. A., Johnson, M., Huang, J. D., Rudolf, M., 2010. Apolipoprotein B-containing lipoproteins in retinal aging and age-related macular degeneration. J Lipid Res 51, 451-467.
Curcio, C. A., Johnson, M., Rudolf, M., Huang, J. D., 2011. The oil spill in ageing Bruch membrane. Br J Ophthalmol 95, 1638-1645.
Ebrahimi, K. B., Handa, J. T., 2011. Lipids, lipoproteins, and age-related macular degeneration. J Lipids 2011, 802059.
Edwards, A. O., Ritter, R., 3rd, Abel, K. J., Manning, A., Panhuysen, C., Farrer, L. A., 2005. Complement factor H polymorphism and age-related macular degeneration. Science 308, 421-424.
Friedman, D. S., O'Colmain, B. J., Munoz, B., Tomany, S. C., McCarty, C., de Jong, P. T., Nemesure, B., Mitchell, P., Kempen, J., 2004. Prevalence of age-related macular degeneration in the United States. Arch Ophthalmol 122, 564-572.
Fritsche, L. G., Chen, W., Schu, M., Yaspan, B. L., Yu, Y., Thorleifsson, G., Zack, D. J., Arakawa, S., Cipriani, V., Ripke, S., Igo, R. P., Jr., Buitendijk, G. H., Sim, X., Weeks, D. E., Guymer, R. H., Merriam, J. E., Francis, P. J., Hannum, G., Agarwal, A., Armbrecht, A. M., Audo, I., Aung, T., Barile, G. R., Benchaboune, M., Bird, A. C., Bishop, P. N., Branham, K. E., Brooks, M., Brucker, A. J., Cade, W. H., Cain, M. S., Campochiaro, P. A., Chan, C. C., Cheng, C. Y., Chew, E. Y., Chin, K. A., Chowers, I., Clayton, D. G., Cojocaru, R., Conley, Y. P., Cornes, B. K., Daly, M. J., Dhillon, B., Edwards, A. O., Evangelou, E., Fagerness, J., Ferreyra, H. A., Friedman, J. S., Geirsdottir, A., George, R. J., Gieger, C., Gupta, N., Hagstrom, S. A., Harding, S. P., Haritoglou, C., Heckenlively, J. R., Holz, F. G., Hughes, G., Ioannidis, J. P., Ishibashi, T., Joseph, P., Jun, G., Kamatani, Y., Katsanis, N., C, N. K., Khan, J. C., Kim, I. K., Kiyohara, Y., Klein, B. E., Klein, R., Kovach, J. L., Kozak, I., Lee, C. J., Lee, K. E., Lichtner, P., Lotery, A. J., Meitinger, T., Mitchell, P., Mohand-Said, S., Moore, A. T., Morgan, D. J., Morrison, M. A., Myers, C. E., Naj, A. C., Nakamura, Y., Okada, Y., Orlin, A., Ortube, M. C., Othman, M. I., Pappas, C., Park, K. H., Pauer, G. J., Peachey, N. S., Poch, O., Priya, R. R., Reynolds, R., Richardson, A. J., Ripp, R., Rudolph, G., Ryu, E., Sahel, J. A., Schaumberg, D. A., Scholl, H. P., Schwartz, S. G., Scott, W. K., Shahid, H., Sigurdsson, H., Silvestri, G., Sivakumaran, T. A., Smith, R. T., Sobrin, L., Souied, E. H., Stambolian, D. E., Stefansson, H., Sturgill-Short, G. M., Takahashi, A., Tosakulwong, N., Truitt, B. J., Tsironi, E. E., Uitterlinden, A. G., van Duijn, C. M., Vijaya, L., Vingerling, J. R., Vithana, E. N., Webster, A. R., Wichmann, H. E., Winkler, T. W., Wong, T. Y., Wright, A. F., Zelenika, D., Zhang, M., Zhao, L., Zhang, K., Klein, M. L., Hageman, G. S., Lathrop, G. M., Stefansson, K., Allikmets, R., Baird, P. N., Gorin, M. B., Wang, J. J., Klaver, C. C., Seddon, J. M., Pericak-Vance, M. A., Iyengar, S. K., Yates, J. R., Swaroop, A., Weber, B. H., Kubo, M., Deangelis, M. M., Leveillard, T., Thorsteinsdottir, U., Haines, J. L., Farrer, L. A., Heid, I. M., Abecasis, G. R., 2013. Seven new loci associated with age-related macular degeneration. Nat Genet.
Gold, B., Merriam, J. E., Zernant, J., Hancox, L. S., Taiber, A. J., Gehrs, K., Cramer, K., Neel, J., Bergeron, J., Barile, G. A., Smith, R. T., Hageman, G. S., Dean, M., Allikmets, R., 2006. Variation in factor B (BF) and complement component 2 (C2) genes is associated with age-related macular degeneration. Nat Genet 38, 458-462.
Hageman, G. S., Anderson, D. H., Johnson, L. V., Hancox, L. S., Taiber, A. J., Hardisty, L. I., Hageman, J. L., Stockman, H. A., Borchardt, J. D., Gehrs, K. M., Smith, R. J., Silvestri, G., Russell, S. A., Klaver, C. C., Barbazetto, I., Chang, S., Yannuzzi, L. A., Barile, G. R., Merriam, J. C., Smith, R. T., Olsh, A. K., Bergeron, J., Zernant, J., Merriam, J. E., Gold, B., Dean, M., Allikmets, R., 2005. A common haplotype in the complement regulatory gene factor H (HF1/CFH) predisposes individuals to age-related macular degeneration. Proc Natl Acad Sci USA 102, 7227-7232.
Hageman, G. S., Luthert, P. J., Victor Chong, N. H., Johnson, L. V., Anderson, D. H., Mullins, R. F., 2001. An integrated hypothesis that considers drusen as biomarkers of immune-mediated processes at the RPE-Bruch's membrane interface in aging and age-related macular degeneration. Prog Retin Eye Res 20, 705-732.
Haines, J. L., Hauser, M. A., Schmidt, S., Scott, W. K., Olson, L. M., Gallins, P., Spencer, K. L., Kwan, S. Y., Noureddine, M., Gilbert, J. R., Schnetz-Boutaud, N., Agarwal, A., Postel, E. A., Pericak-Vance, M. A., 2005. Complement factor H variant increases the risk of age-related macular degeneration. Science 308, 419-421.
Houben, A. J., Moolenaar, W. H., 2011. Autotaxin and LPA receptor signaling in cancer. Cancer Metastasis Rev 30, 557-565.
Johnson, L. V., Forest, D. L., Banna, C. D., Radeke, C. M., Maloney, M. A., Hu, J., Spencer, C. N., Walker, A. M., Tsie, M. S., Bok, D., Radeke, M. J., Anderson, D. H., 2011. Cell culture model that mimics drusen formation and triggers complement activation associated with age-related macular degeneration. Proc Natl Acad Sci USA 108, 18277-18282.
Johnson, P. T., Betts, K. E., Radeke, M. J., Hageman, G. S., Anderson, D. H., Johnson, L. V., 2006. Individuals homozygous for the age-related macular degeneration risk-conferring variant of complement factor H have elevated levels of CRP in the choroid. Proc Natl Acad Sci USA 103, 17456-17461.
Kensil, C. R., Dennis, E. A., 1981. Alkaline hydrolysis of phospholipids in model membranes and the dependence on their state of aggregation. Biochemistry 20, 6079-6085.
Klaver, C. C., Kliffen, M., van Duijn, C. M., Hofman, A., Cruts, M., Grobbee, D. E., van Broeckhoven, C., de Jong, P. T., 1998. Genetic association of apolipoprotein E with age-related macular degeneration. Am J Hum Genet 63, 200-206.
Klein, R. J., Zeiss, C., Chew, E. Y., Tsai, J. Y., Sackler, R. S., Haynes, C., Henning, A. K., SanGiovanni, J. P., Mane, S. M., Mayne, S. T., Bracken, M. B., Ferris, F. L., Ott, J., Barnstable, C., Hoh, J., 2005. Complement factor H polymorphism in age-related macular degeneration. Science 308, 385-389.
Lachmann, P. J., 2010. Preparing serum for functional complement assays. J Immunol Methods 352, 195-197.
Laine, M., Jarva, H., Seitsonen, S., Haapasalo, K., Lehtinen, M. J., Lindeman, N., Anderson, D. H., Johnson, P. T., Jarvela, I., Jokiranta, T. S., Hageman, G. S., Immonen, I., Meri, S., 2007. Y402H polymorphism of complement factor H affects binding affinity to C-reactive protein. J Immunol 178, 3831-3836.
Li, C. M., Clark, M. E., Chimento, M. F., Curcio, C. A., 2006. Apolipoprotein localization in isolated drusen and retinal apolipoprotein gene expression. Invest Ophthalmol Vis Sci 47, 3119-3128.
Mold, C., Gewurz, H., Du Clos, T. W., Regulation of complement activation by C-reactive protein, Immunopharmacology, 42 (1999) 23-30.
Moolenaar, W. H., Hla, T., 2012. SnapShot: Bioactive lysophospholipids. Cell 148, 378-378 e372.
Mullins, R. F., Russell, S. R., Anderson, D. H., Hageman, G. S., 2000. Drusen associated with aging and age-related macular degeneration contain proteins common to extracellular deposits associated with atherosclerosis, elastosis, amyloidosis, and dense deposit disease. FASEB J 14, 835-846.
Neale, B. M., Eagerness, J., Reynolds, R., Sobrin, L., Parker, M., Raychaudhuri, S., Tan, P. L., Oh, E. C., Merriam, J. E., Souied, E., Bernstein, P. S., Li, B., Frederick, J. M., Zhang, K., Brantley, M. A., Jr., Lee, A. Y., Zack, D. J., Campochiaro, B., Campochiaro, P., Ripke, S., Smith, R. T., Barile, G. R., Katsanis, N., Allikmets, R., Daly, M. J., Seddon, J. M., 2010. Genome-wide association study of advanced age-related macular degeneration identifies a role of the hepatic lipase gene (LIPC). Proc Natl Acad Sci USA 107, 7395-7400.
Okemefuna, A. I., Nan, R., Miller, A., Gor, J., Perkins, S. J., 2010. Complement factor H binds at two independent sites to C-reactive protein in acute phase concentrations. J Biol Chem 285, 1053-1065.
Rivera, A., Fisher, S. A., Fritsche, L. G., Keilhauer, C. N., Lichtner, P., Meitinger, T., Weber, B. H., 2005. Hypothetical LOC387715 is a second major susceptibility gene for age-related macular degeneration, contributing independently of complement factor H to disease risk. Hum Mol Genet 14, 3227-3236.
Souied, E. H., Benlian, P., Amouyel, P., Feingold, J., Lagarde, J. P., Munnich, A., Kaplan, J., Coscas, G., Soubrane, G., 1998. The epsilon4 allele of the apolipoprotein E gene as a potential protective factor for exudative age-related macular degeneration. Am J Ophthalmol 125, 353-359.
St George-Hyslop, P., Haines, J., Rogaev, E., Mortilla, M., Vaula, G., Pericak-Vance, M., Foncin, J. F., Montesi, M., Bruni, A., Sorbi, S., Rainero, I., Pinessi, L., Pollen, D., Polinsky, R., Nee, L., Kennedy, J., Macciardi, F., Rogaeva, E., Liang, Y., Alexandrova, N., Lukiw, W., Schlumpf, K., Tanzi, R., Tsuda, T., Farrer, L., Cantu, J. M., Duara, R., Amaducci, L., Bergamini, L., Gusella, J., Roses, A., Crapper McLachlan, D., et al., 1992. Genetic evidence for a novel familial Alzheimer's disease locus on chromosome 14. Nat Genet 2, 330-334.
Volanakis, J. E., Narkates, A. J., 1981. Interaction of C-reactive protein with artificial phosphatidylcholine bilayers and complement. J Immunol 126, 1820-1825.
Volanakis, J. E., Wirtz, K. W., 1979. Interaction of C-reactive protein with artificial phosphatidylcholine bilayers. Nature 281, 155-157.
Wang, L., Clark, M. E., Crossman, D. K., Kojima, K., Messinger, J. D., Mobley, J. A., Curcio, C. A., 2010. Abundant lipid and protein components of drusen. PLoS One 5, e10329.
Wang, L., Li, C. M., Rudolf, M., Belyaeva, O. V., Chung, B. H., Messinger, J. D., Kedishvili, N. Y., Curcio, C. A., 2009. Lipoprotein particles of intraocular origin in human Bruch membrane: an unusual lipid profile. Invest Ophthalmol Vis Sci 50, 870-877.
Yates, J. R., Sepp, T., Matharu, B. K., Khan, J. C., Thurlby, D. A., Shahid, H., Clayton, D. G., Hayward, C., Morgan, J., Wright, A. F., Armbrecht, A. M., Dhillon, B., Deary, I. J., Redmond, E., Bird, A. C., Moore, A. T., 2007. Complement C3 variant and the risk of age-related macular degeneration. N Engl J Med 357, 553-561.

1. A method of treating a subject suffering from wet acute macular degeneration which comprises administering to the subject an amount of a modified alpha cyclodextrin effective to treat the subject, wherein the modified alpha cyclodextrin binds to bioactive lipids which accumulate in the subject's eye and are characterized by the presence of a single chain of fatty acids.

2. The method of claim 1, wherein the binding of the modified alpha cyclodextrin to the bioactive lipids facilitates clearance of the lipids from the subject's eye.

3. The method of claim 1, wherein the modified alpha cyclodextrin is selected from the group consisting of hydroxypropyl alpha cyclodextrin, hydroxybutyl alpha cyclodextrin, sulfobutyl alpha cyclodextrin, sulfopropyl alpha cyclodextrin, carboxyethyl alpha cyclodextrin, succinyl alpha cyclodextrin and succinylhydroxypropyl alpha cyclodextrin.

4. The method of claim 1 or 2, wherein the modified alpha cyclodextrin is selected from the group consisting of 2-hydroxypropyl alpha cyclodextrin, 2-hydroxybutyl alpha cyclodextrin and 2-succinylhydroxypropyl alpha cyclodextrin.

5. The method of claim 1, wherein the modified alpha cyclodextrin is 2-hydroxypropyl alpha cyclodextrin.

6. The method of claim 1, wherein the bioactive lipids are lysophospolipids.

7. The method of claim 1, wherein the modified alpha cyclodextrin is administered as a monotherapy.

8. The method of claim 1, which further comprises coadministering a second therapeutic agent for treating acute macular degeneration.

9. The method of claim 8, wherein the second therapeutic agent is selected from the group consisting of ranibizumab, bevacizumab, pegaptanib sodium, aflibercept and verteporfin.

10. The method of claim 1, wherein the administering comprises administering eyedrops to the subject.

11. The method of claim 1, wherein the administering comprises intravitreally injecting the modified alpha cyclodextrin.

12. A method of treating a subject suffering from a cancer associated with lipid accumulation which comprises administering to the subject an amount of a modified alpha cyclodextrin effective to treat the subject, wherein the modified alpha cyclodextrin binds to the lipid.

13. The method of claim 12, wherein the lipid is characterized by the presence of a single chain of fatty acids.

14. The method of claim 12, wherein the modified alpha cyclodextrin is selected from the group consisting of hydroxypropyl alpha cyclodextrin, hydroxybutyl alpha cyclodextrin, sulfobutyl alpha cyclodextrin, sulfopropyl alpha cyclodextrin, carboxyethyl alpha cyclodextrin, succinyl alpha cyclodextrin and succinylhydroxypropyl alpha cyclodextrin.

15. The method of claim 12, wherein the modified alpha cyclodextrin is selected from the group consisting of 2-hydroxypropyl alpha cyclodextrin, 2-hydroxybutyl alpha cyclodextrin and 2-succinylhydroxypropyl alpha cyclodextrin.

16. The method of claim 12, wherein the modified alpha cyclodextrin is 2-hydroxypropyl alpha cyclodextrin.

17. The method of claim 12, wherein the lipids comprise lysophospolipids.

18. (canceled)

19. The method of claim 12, which further comprises coadministering a second therapeutic agent for treating cancer.

20. The method of claim 19, wherein the second therapeutic agent is selected from the group consisting of temozolomide, a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, camptothecins, bleomycin, doxorubicin, idarubicin, daunorubicin, dactinomycin, plicamycin, mitoxantrone, L-asparaginase, epirubicin, 5-fluorouracil, taxanes such as docetaxel and paclitaxel, leucovorin, levamisole, irinotecan, estramustine, etoposide, nitrogen mustards, 1,3-bis(2-chloroethyl)-1-nitrosourea (BCNU), nitrosoureas such as carmustine and lomustine, vinca alkaloids such as vinblastine, vincristine and vinorelbine, platinum complexes such as cisplatin, carboplatin and oxaliplatin, imatinib mesylate, hexamethylmelamine, topotecan, tyrosine kinase inhibitors, tyrphostins, herbimycin A, genistein, erbstatin and lavendustin A.

21. A method of treating a subject suffering from atherosclerosis associated with lipid accumulation which comprises administering to the subject an amount of a modified alpha cyclodextrin effective to treat the subject, wherein the modified alpha cyclodextrin binds to the lipid.

22-27. (canceled)
